(19) United States
(12) Patent Application Publication
(10) Pub. No.: US 2021/0113496 A1
(43) Pub. Date: Apr. 22, 2021

(54) Methods of Treating Excitotoxicity Disorders

(71) Applicant: HORIZON ORPHAN LLC, Lake Forest, IL (US)

(72) Inventors: Todd C. Zankel, San Francisco, CA (US); Amanda Anne Ko, San Rafael, CA (US); Sara Louise Isbell, Novato, CA (US)

(21) Appl. No.: 17/050,041

(22) PCT Filed: Apr. 25, 2019
(86) PCT No.: PCT/US19/29069

§ 371 Date: Oct. 23, 2020

Related U.S. Application Data
(60) Provisional application No. 62/662,505, filed on Apr. 25, 2018.

(51) Int. Cl. A61K 31/145; A61K 31/635; A61P 25/28

(52) U.S. Cl. CPC A61K 31/145; A61P 25/28; A61K 31/635

(57) ABSTRACT

The present disclosure relates in general to methods for the treatment of excitotoxicity disorders, using compositions comprising cysteamine or cystamine or salts or derivatives thereof in combination with an agent that inhibits or blocks glutamate/cystine antiporter xc⁻.

### FIELD OF THE INVENTION

The present disclosure relates in general to methods for the treatment of excitotoxic disease, including neurodegenerative diseases, using compositions comprising cysteamine or cystamine or salts or derivatives thereof in combination with an agent that blocks the glutamate/cystine antiporter x_{c}⁻.

### BACKGROUND OF THE INVENTION

Excitotoxicity disorders affect the central nervous and peripheral nervous systems and can lead to progressive neurodegeneration. Excitotoxicity results from excess glutamate being secreted by various cells, including immune cells and neurons, in the brain. Glutamate is the primary excitatory neurotransmitter in the mammalian nervous system. Three types of glutamate-gated ion channel receptors transduce postsynaptic signals, including α-amino-3-hydroxy-5-methyl-4-isoxazole propionic acid receptor (AMPAR), kainate receptor, and N-methyl-D-aspartate receptor (NMDAR).

Resting extracellular glutamate concentrations under physiological conditions are usually in the low micromolar range. During synaptic transmission the levels increase briefly to reach mM concentrations (Clements et al., Science. 258:1498-1501, 1992). Levels of extracellular glutamate are regulated after synaptic glutamate release by uptake processes and intracellular metabolism of glutamate to glutamine by glutamine synthetase. Glutamine can passively diffuse to the presynaptic button where it is recycled into glutamate by glutaminase (Danbolt N C. Prog Neurobiol. 65:1-105 2001). Prolonged glutamate signaling leads to a type of toxicity characterized by elevated mitochondrial activity, gradual glutathione (GSH) depletion, oxidative stress and apoptosis (Ratan et al., J Neurochem 62:376-379, 1994; Shih et al., J Neurosci. 26:10514-523, 2006).

Glutathione is a tripeptide made of glutamate-cysteine-glycine and is an important combatant of oxidative stress in the brain. GSH is synthesized after sulfur amino acid cysteine is oxidized to cystine, the cystine is then taken up via the glutamate:cystine exchange transporter x_{c}⁻, converted back to two cysteine molecules and the cysteine is incorporated into glutathione. The x_{c}⁻ transporter, also called the x_{c}⁻ antiporter, or xCT, is a Na+-independent cystine-glutamate exchange system that takes up cystine and exports glutamate from the cell in a 1:1 exchange ratio (Shih et al., supra).

Glutathione-based antioxidant systems exhibit redundancy with a system that includes such components as thioredoxin, thioredoxin reductase, TRP14, peroxiredoxin, nicotinamide nucleotide transhydrogenase and reduced nicotinamide adenine dinucleotide cofactors. Sulfur amino acids are also a key feature of this second anti-oxidant network, which, therefore, also depends on xCT.

Pharmacologically, application of small thiol molecules has been demonstrated to rescue deficits in antioxidant capacity, including complete loss of the GSH-based system. The basis for this effect is unclear.

### SUMMARY OF THE INVENTION

The present invention relates to treatment of a excitotoxicity diseases or disorders, such as Huntington's Disease, Parkinson's disease, ischemia, Amyotrophic Lateral Sclerosis or Alzheimer's Disease, using a cysteamine composition (e.g., cysteamine or a pharmaceutically acceptable salt thereof or cystamine or a pharmaceutically acceptable salt thereof or cysteamine analogs) in combination with an agent that blocks the activity of the x_{c}⁻ cystine/glutamate transporter. The combination of the compositions increases glutathione synthesis in the cell while blocking glutamate release from the cell by the x_{c}⁻ transporter.

In various embodiments, the disclosure provides a method for treating a subject having an excitotoxicity disorder comprising administering an effective amount of a cysteamine composition in combination with an agent that blocks activity of glutamate/cystine antiporter x_{c}⁻.

In various embodiments, the disclosure provides a method for slowing the degeneration of neurons in a subject comprising administering an effective amount of a cysteamine composition in combination with an agent that blocks glutamate/cystine antiporter x_{c}⁻.

Also contemplated herein is a method for treating or ameliorating glutamate cytotoxicity in a subject comprising administering an effective amount of a cysteamine composition in combination with an agent that blocks glutamate/cystine antiporter x_{c}⁻.

In various embodiments, the administering reduces neuronal glutamate toxicity.

In various embodiments, the agent that inhibits x_{c}⁻ activity is selected from the group consisting of sulfasalazine, 4-s-carboxyphenylglycine, 4-s-sulfonylphenylglycine, sorafenib, erastin, and [(R,S)-4-[4′-carboxyphenyl]-phenylglycine. In various embodiments, the agent is sulfasalazine.

In various embodiments, the excitotoxicity disorder is selected from the group consisting of spinal cord injury, stroke, traumatic brain injury, chronic traumatic encephalopathy (CTE), hearing loss, neurodegenerative diseases, multiple sclerosis, Alzheimer's disease, amyotrophic lateral sclerosis (ALS), Parkinson's disease, Huntington's disease, concussion, and CNS depressant withdrawal syndrome.

It is further contemplated that the methods of the disclosure can be carried out using a soluble, diffusible small thiol compound, e.g., that can cross the blood brain barrier, in order to treat a subject having an excitotoxicity disorder, slow the degeneration of neurons in a subject and/or treat or ameliorate glutamate cytotoxicity in a subject. Exemplary small thiol compounds include, but are not limited to, thiomandelic acid, Captopril, Thiorphan, N-acetylcysteine, 2,3-dimercaptosuccinate, 2,3-dimercaprol, penicillamine, glutathione, cysteine, homocysteine, Zofenoprilat, Tiopronin, pantetheine, coenzyme A, amifostine, WR-1065, thiophenol, thioacetic acid, 2-mercaptoethanol, dithiothreitol, dithioerythritol, 2-mercaptoindole, and disulfides, mixed or symmetrical, of any of the above.

In various embodiments, the amount of cysteamine composition administered is from about 1 to about 50 mg/kg/day or from about 2 mg/kg/day to about 25 mg/kg/day. In various embodiments, the cysteamine composition, e.g., cysteamine or a pharmaceutically acceptable salt thereof or cystamine or a pharmaceutically acceptable salt thereof, is administered in a total daily dose of about 2 to 10 mg/kg, about 5 to 15 mg/kg, about 15 to 25 mg/kg, about 15 to 20 mg/kg or about 10 to 20 mg/kg, over one, two or three doses daily. In various embodiments, the cysteamine composition is cysteamine or a pharmaceutically acceptable salt thereof or cystamine or a pharmaceutically acceptable salt thereof which is administered in a total daily dose of approximately 200, 300, 400, 500, 600, 700, 800, 900, 1000, 1100, 1200, 1300, 1400 or 1500 mg per day in one, two or three doses.

In various embodiments, the amount of agent that inhibits the x_{c}⁻ transporter is administered at a dose of from about 10 to about 100 mg/kg/day or from about 200 mg to 3 grams/day. In various embodiments, the amount of agent that inhibits the xc− transporter is administered at a dose of from about 10 to 1000 mg/kg/day, from about 10 to 500 mg/kg/day, from about 500 to 2500 mg/kg/day, or from about 1000 to 3000 mg/kg/day. In various embodiments, the inhibitor of x_{c}⁻ activity is selected from the group consisting of sulfasalazine, 4-s-carboxyphenylglycine, 4-s-sulfonylphenylglycine, sorafenib, erastin, and [(R,S)-4-[4′-carboxyphenyl]-phenylglycine. In various embodiments, the inhibitor of x_{c}⁻ activity is sulfasalazine.

In various embodiments, glutathione levels in the subject are increased. In various embodiments, glutathione levels in the subject are increased by about 5, 10, 15, 20, 25, 30, 40, 50, 60, 70, 80, 90 or 100% or more.

In various embodiments, striatal neuron damage is reduced in the subject compared to subjects not receiving the cysteamine composition and x_{c}⁻ inhibitor.

In various embodiments, the cysteamine composition is given prior to the x_{c}⁻ inhibitor, concurrently with the x_{c}⁻ inhibitor or after the x_{c}⁻ inhibitor.

In various embodiments, the administering improves one or more symptoms total motor score, mobility, cognitive ability, or other symptom of an excitotoxicity disorder. In various embodiments, the one or more symptom includes total motor score, mobility, cognitive ability, or other symptom of an excitotoxicity disorder.

In various embodiments, the cysteamine composition is in a delayed release or extended release formulation. In various embodiments, the delayed release composition is enterically coated. For example, the coating can be selected from the group consisting of polymerized gelatin, shellac, methacrylic acid copolymer type CNF, cellulose butyrate phthalate, cellulose hydrogen phthalate, cellulose proprionate phthalate, polyvinyl acetate phthalate (PVAP), cellulose acetate phthalate (CAP), cellulose acetate trimellitate (CAT), hydroxypropyl methylcellulose phthalate, hydroxypropyl methylcellulose acetate, dioxypropyl methylcellulose succinate, carboxymethyl ethylcellulose (CMEC), hydroxypropyl methylcellulose acetate succinate (HPMCAS), and acrylic acid polymers and copolymers, typically formed from methyl acrylate, ethyl acrylate, methyl methacrylate and/or ethyl methacrylate with copolymers of acrylic and methacrylic acid esters. The composition can be administered orally or parenterally. Additional enteric coatings and formulations contemplated herein are discussed further in the Detailed Description.

In some embodiments, the delayed release formulation comprises an enteric coating that releases the cysteamine or cystamine when the formulation reaches the small intestine or a region of the gastrointestinal tract of a subject in which the pH is greater than about pH 4.5. In various embodiments, the formulation releases at a pH of about 4.5 to 6.5, 4.5 to 5.5, 5.5 to 6.5 or about pH 4.5, 5.0, 5.5, 6.0 or 6.5.

In various embodiments, the cysteamine composition, e.g., cysteamine, cystamine or pharmaceutically acceptable salt thereof, is formulated in a tablet or capsule which is enterically coated.

In various embodiments, the cysteamine composition comprises a pharmaceutically acceptable carrier. It is further contemplated that the cysteamine or cystamine or pharmaceutically acceptable salts thereof are formulated as a sterile pharmaceutical composition.

In various embodiments, the administration results in a slower progression in decline of total motor score compared to a subject not receiving the treatment herein. In some embodiments, the slower progression is a result in a decreased change in one or more motor scores selected from the group consisting of chorea subscore, balance and gait subscore, hand movements subscore, eye movement subscore and maximal dystonia subscore, bradykinesia assessment.

In certain embodiments, alteration in one or more symptoms in patients receiving cysteamine composition and x_{c}⁻ inhibitor as described herein is shown to be beneficial by at least 10%, 20%, 25%, 30%, 35%, 40%, 45%, 50%, 55%, 60%, 65%, 70%, 75% or more compared to baseline assessment of the symptom. In certain embodiments, the rate of progression or decline in total motor score is slowed, by at least 20%, 25%, 30%, 35%, 40%, 45%, 50%, 55%, 60%, 65%, 70%, 75% or more. Measurement may be performed using techniques known to those in the art, such as the Unified Huntington Disease Rating Scale (UHDRS), Bradykinesia Ratings Scale, and/or Lindop Parkinson's Assessment Scale (LPAS).

Additional indicia of a slower decline in neurological symptoms of an excitatory disorder are measured using change from baseline in one or more of the following parameters: using standardized tests for (i) functional assessment (e.g., UHDRS Total Functional Capacity, LPAS, Independence Scale); (ii) neuropsychological assessment (e.g., UHDRS Cognitive Assessment, Mattis Dementia Rating Scale, Trail Making Test A and B, Figure Cancellation Test, Hopkins Verbal Learning Test, Articulation Speed Test); (iii) psychiatric assessment (UHDRS Behavioral Assessment, Montgomery and Asberg Depression Rating Scale) and (iv) cognitive assessment (e.g., Dementia Outcomes Measurement Suite (DOMS)).

In certain embodiments, the symptoms are assayed at 6 months, 12 months, 18 months or 2 years or more after administration.

The disclosure also provides a method for slowing the progression of brain and striatal atrophies in a subject suffering from an excitotoxicity disease or disorder comprising administering to a subject in need thereof a composition comprising cysteamine composition in a total daily dose of approximately 200 to 1500 mg, or approximately 200, 300, 400, 500, 600, 700, 800, 900, 1000, 1100, 1200, 1300, 1400 or 1500 mg, given in two doses, in combination with an agent that inhibits x_{c}⁻ transporter.

In various embodiments, the subject suffering from an excitotoxicity disease or disorder suffers from Huntington's disease. In various embodiments, it is contemplated that the method herein is useful to treat any stage of Huntington's disease (stages 1-5), including early stages, such as stage 1 or stage 2, intermediate stages, such as stage 3 and stage 4, and advanced Huntington's Disease, such as stage 5 HD. Further discussion of the stages of HD are provided in the Detailed Description.

In various embodiments, the excitotoxicity disorder is Alzheimer's Disease.

It is contemplated that there may be a certain period during treatment where the dose of cysteamine composition and x_{c}⁻ inhibitor needs to be varied during a ramp up or ramp down phase.

In various embodiments, the total daily dose of cysteamine composition is between 200 to 2000 mg, 500 to 2000 mg, 200 to 1000 mg, 750 to 1750 mg, 1000 to 1500 mg, or may range between any two of the foregoing values. In various embodiments, the total daily dose of cysteamine composition, including cysteamine or a pharmaceutically acceptable salt thereof or cystamine or a pharmaceutically acceptable salt thereof, is 200, 300, 400, 500, 600, 700, 800, 900, 1000, 1100, 1200, 1300, 1400, 1500, 1600, 1700, 1800, 1900 or 2000 mg per day. It is contemplated that any of the foregoing doses is administered twice daily. It is further contemplated that any of the foregoing doses is administered in two equal doses daily.

Also contemplated herein is administration of the cysteamine composition at a daily dose ranging from about 10 mg/kg to about 250 mg/kg, or from about 100 mg/kg to about 250 mg/kg, or from about 60 mg/kg to about 100 mg/kg or from about 50 mg/kg to about 90 mg/kg, or from about 30 mg/kg to about 80 mg/kg, or from about 20 mg/kg to about 60 mg/kg, or from about 10 mg/kg to about 50 mg/kg. Further, the effective dose may be about 0.5 mg/kg, 1 mg/kg, 2, mg/kg, 3 mg/kg, 4 mg/kg, 5 mg/kg, 10 mg/kg, 15 mg/kg, 20 mg/kg/25 mg/kg, 30 mg/kg, 35 mg/kg, 40 mg/kg, 45 mg/kg, 50 mg/kg, 55 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, 225 mg/kg, 250 mg/kg, 275 mg/kg, 300 mg/kg, 325 mg/kg, 350 mg/kg, 375 mg/kg, 400 mg/kg, 425 mg/kg, 450 mg/kg, 475 mg/kg, 500 mg/kg, 525 mg/kg, 550 mg/kg, 575 mg/kg, 600 mg/kg, 625 mg/kg, 650 mg/kg, 675 mg/kg, 700 mg/kg, 725 mg/kg, 750 mg/kg, 775 mg/kg, 800 mg/kg, 825 mg/kg, 850 mg/kg, 875 mg/kg, 900 mg/kg, 925 mg/kg, 950 mg/kg, 975 mg/kg or 1000 mg/kg, or may range between any two of the foregoing values. In some embodiments, the cysteamine composition is administered at a total daily dose of from approximately 0.25 g/m² to 4.0 g/m² body surface area, about 0.5-2.0 g/m² body surface area, or 1-1.5 g/m² body surface area, or 1-1.95 g/m² body surface area, or 0.5-1 g/m² body surface area, or about 0.7-0.8 g/m² body surface area, or about 1.35 g/m² body surface area, or about 1.3 to about 1.95 grams/m²/day, or about 0.5 to about 1.5 grams/m2/day, or about 0.5 to about 1.0 grams/m²/day, e.g., at least about 0.3, 0.4, 0.5, 0.6, 0.7, 0.8, 0.9, 1.0, 1.1, 1.2, 1.3, 1.4, 1.5, 1.6, 1.7, 1.8, 1.9 or 2 g/m², or up to about 0.8, 0.9, 1.0, 1.1, 1.2, 1.3, 1.4, 1.5, 1.6, 1.7, 1.8, 1.9, 2.0, 2.2, 2.5, 2.7, 3.0, 3.25, 3.5 or 3.75 g/m² or may range between any two of the foregoing values.

Aspects of the disclosure that are described herein as methods (especially methods that involve treatment) can alternatively be described as (medical) uses of a cysteamine composition, e.g., cysteamine or a pharmaceutically acceptable salt thereof or cystamine or a pharmaceutically acceptable salt thereof. For example, in one variation, described herein the use of a cysteamine composition to treat an excitatotry disease or disorder.

In the treatment methods (or uses) described herein, the methods optionally comprise administering an adjunct therapy to the subject in combination with the cysteamine composition and x_{c}⁻ inhibitor. In some embodiments, the adjunct therapy is selected from the group consisting of antipsychotics, antidepressants, vesicular monoamine transporter (VMAT)-inhibitors such as tetrabenazine, dopamine inhibitors, laquinimod, CNS-immunomodulators, neuroprotective factors, BDNF and agents that upregulate BDNF, ampakines, positive modulators of AMPA-type glutamate receptors, activators of BDNF receptor TrkB and gene therapy.

Antidepressants include: SSRI antidepressants, such as fluoxetine, citalopram and paroxetine, tricyclic antidepressants, such as amitriptyline, other types of antidepressants, including mirtazapine, duloxetine and venlafaxine.

Antipsychotic medication includes risperidone, olanzapine, aripiprazole, tiapride and quetiapine, benzodiazepines, such as clonazepam and diazepam, and mood stabilizers, such as carbamazepine.

In some embodiments, the methods (or uses) described herein further comprise administering a further therapeutic agent selected from the group consisting of tetrabenazine, laquinimod, BDNF, ampakines, fluoxetine, citalopram, paroxetine, amitriptyline, mirtazapine, duloxetine, venlafaxine, risperidone, olanzapine, aripiprazole, tiapride, quetiapine, clonazepam diazepam and carbamazepine.

In various embodiments, the cysteamine composition and/or x_{c}⁻ inhibitor is administered parenterally or orally. In various embodiments, the therapeutic agent further comprises a pharmaceutically acceptable carrier. It is further contemplated that the cysteamine composition and x_{c}⁻ inhibitor are formulated as sterile pharmaceutical compositions, either alone or in combination.

In various embodiments, the methods herein comprise administering cysteamine or a pharmaceutically acceptable salt thereof. In some embodiments, the salt is cysteamine bitartrate or cysteamine hydrochloride. In various embodiments, the cysteamine bitartrate or cysteamine hydrochloride is in a delayed release formulation.

With respect to any combination treatments described herein, the cysteamine composition can be administered simultaneously with the other active agents, which may be in admixture with the agent or may be in a separate composition. In various embodiments, the agent is an inhibitor of the x_{c}⁻ transporter. Each composition preferably includes a pharmaceutically acceptable diluent, adjuvant, or carrier. When the agents are separately administered, they may be administered in any order.

In another aspect, described herein is a method of increasing levels of brain derived neurotrophic factor (BDNF) activity in a brain or neuronal cell comprising contacting the cell with a cysteamine composition in combination with a x_{c}⁻ inhibitor, optionally with another agent, in an amount effective to increase BDNF activity in the cell. In some embodiments, increased levels of BDNF is demonstrated when compared to levels before administration described herein.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document.

In addition to the foregoing, the invention includes, as an additional aspect, all embodiments of the invention narrower in scope in any way than the variations defined by specific paragraphs above. For example, certain aspects of the invention that are described as a genus, and it should be understood that every member of a genus is, individually, an aspect of the invention. Also, aspects described as a genus or selecting a member of a genus, should be understood to embrace combinations of two or more members of the genus. Although the applicant(s) invented the full scope of the invention described herein, the applicants do not intend to paragraph subject matter described in the prior art work of others. Therefore, in the event that statutory prior art within the scope of a paragraph is brought to the attention of the applicant(s) by a Patent Office or other entity or individual, the applicant(s) reserve the right to exercise amendment rights under applicable patent laws to redefine the subject matter of such a paragraph to specifically exclude such statutory prior art or obvious variations of statutory prior art from the scope of such a paragraph. Variations of the invention defined by such amended paragraphs also are intended as aspects of the invention.

### BRIEF DESCRIPTION OF THE DRAWINGS

FIG. 1A illustrates the effects of cysteamine on cell viability after induced glutamate toxicity and FIG. 1B illustrates the effects of cysteamine in combination with other agents on glutamate cytotoxicity.

FIG. 2A shows the effects of cysteamine on cell proliferation after culture of neurons in glutamate, as depicted in relative absorbance units (RAU) and FIG. 2B shows proliferation by cell number.

FIG. 3 shows the effects of cysteamine in neurons after 24 and 48 hours of culture with glutamate.

### DETAILED DESCRIPTION

The present disclosure relates to the treatment of excitotoxicity disorders, including neurodegenerative diseases, such as Huntington's Disease, Parkinson's disease, ischemia or Alzheimer's disease, using a composition in combination with an agent that inhibits the glutamate/cysteine antiporter x_{c}⁻.

Glutamate is a competitive inhibitor of cystine import by x_{c}⁻, also called xCT. Blockade of cystine entry into a cell by x_{c}⁻ quickly weakens the cell's sulfur-based antioxidant systems, which are required to cope with increased metabolic activities resulting from chronic glutamate excitation. The reaction products of cystine and cysteamine in the extracellular space enter the cell through import pathways that are independent of x_{c}⁻, circumventing glutamate blockade of cystine import, obligate glutamate export upon cystine import, and diminished x_{c}⁻ expression. The administration of a combination of a cysteamine composition plus an agent that inhibits glutamate transport out of the cell will diminish sensitivity of neuronal cells to glutamate toxicity, such as those found in neurodegenerative diseases, and improve outcomes of subjects suffering from an excitotoxicity disorder.

### Definitions

As used herein and in the appended claims, the singular forms “a,” “and,” and “the” include plural referents unless the context clearly dictates otherwise. Thus, for example, reference to “a derivative” includes a plurality of such derivatives and reference to “a patient” includes reference to one or more patients and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and products, the exemplary methods, devices and materials are described herein.

The documents discussed above and throughout the text are provided solely for their disclosure prior to the filing date of the present application. Nothing herein is to be construed as an admission that the inventors are not entitled to antedate such disclosure by virtue of prior disclosure. Each document is incorporated by reference in its entirety with particular attention to the disclosure for which it is cited.

The following references provide one of skill with a general definition of many of the terms used in this disclosure: Singleton, et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger, et al. (eds.), Springer Verlag (1991); and Hale and Marham, THE HARPER COLLINS DICTIONARY OF BIOLOGY (1991).

As used herein a “cysteamine composition” or “cysteamine product” refers generally to cysteamine or a pharmaceutically acceptable salt thereof or cystamine or a pharmaceutically acceptable salt thereof, including a biologically active metabolite or derivative thereof, structural analogs of cysteamine or cystamine, or combination of cysteamine and cystamine, and includes cysteamine or cystamine salts, esters, amides, alkylate compounds, prodrugs, analogs, phosphorylated compounds, sulfated compounds, nitrosylated and glycosylated compounds or other chemically modified forms thereof (e.g., chemically modified forms prepared by labeling with radionucleotides or enzymes and chemically modified forms prepared by attachment of polymers such as polyethylene glycol). Thus, the cysteamine or cystamine composition can be administered in the form of a pharmacologically acceptable salt, ester, amide, prodrug or analog or as a combination thereof. In various embodiments, the cysteamine product includes cysteamine, cystamine or derivatives thereof. In any of the embodiments described herein, a cysteamine product may optionally exclude N-acetylcysteine.

As used herein “an inhibitor of the x_{c}⁻ transporter”, “inhibitor of x_{c}⁻”, “inhibitor of x_{c}⁻ activity” or “x_{c}⁻ inhibitor” refers to an agent that can inhibit or block the activity of the x_{c}⁻ protein to transport cystine into a cell and transport glutamate out of a cell. Exemplary agents that inhibit x_{c}⁻ activity include, but are not limited to, sulfasalazine, 4-s-carboxyphenylglycine, 4-s-sulfonylphenylglycine, sorafenib, erastin, and [(R,S)-4-[4′-carboxyphenyl]-phenylglycine.

As used herein, a “therapeutically effective amount” or “effective amount” refers to that amount of a cysteamine composition or cysteamine product, e.g., cysteamine or a pharmaceutically acceptable salt thereof or cystamine or a pharmaceutically acceptable salt thereof, and/or an agent that inhibits the glutamate/cystine antiporter x_{c}⁻, and/or diffusible small thiol compound, sufficient to result in amelioration of symptoms, for example, treatment, healing, prevention or amelioration of the relevant medical condition, or an increase in rate of treatment, healing, prevention or amelioration of such conditions, typically providing a statistically significant improvement in the treated patient population. When referencing an individual active ingredient, administered alone, a therapeutically effective dose refers to that ingredient alone. When referring to a combination, a therapeutically effective dose refers to combined amounts of the active ingredients that result in the therapeutic effect, whether administered in combination, including serially or simultaneously. In various embodiments, a therapeutically effective amount of the cysteamine product in combination with an agent that inhibits the glutamate/cystine transporter x_{c}⁻ ameliorates one or more symptoms associated with various neurodegenerative diseases, including but not limited to, bradykinesia, dystonia, motor deficiencies, cognitive dysfunction, and psychiatric episodes, including depression.

“Treatment” refers to prophylactic treatment or therapeutic treatment. In certain embodiments, “treatment” refers to administration of a compound or composition to a subject for therapeutic or prophylactic purposes.

A “therapeutic” treatment is a treatment administered to a subject who exhibits signs or symptoms of pathology for the purpose of diminishing or eliminating those signs or symptoms. The signs or symptoms may be biochemical, cellular, histological, functional or physical, subjective or objective.

A “prophylactic” treatment is a treatment administered to a subject who does not exhibit signs of a disease or exhibits only early signs of the disease, for the purpose of decreasing the risk of developing pathology. The compounds or compositions of the disclosure may be given as a prophylactic treatment to reduce the likelihood of developing a pathology or to minimize the severity of the pathology, if developed.

“Diagnostic” means identifying the presence, extent and/or nature of a pathologic condition. Diagnostic methods differ in their specificity and selectivity. While a particular diagnostic method may not provide a definitive diagnosis of a condition, it suffices if the method provides a positive indication that aids in diagnosis.

“Pharmaceutical composition” refers to a composition suitable for pharmaceutical use in a subject animal, including humans and mammals. In various embodiments, a pharmaceutical composition comprises a therapeutically effective amount of a cysteamine product or diffusible small thiol compound, optionally another biologically active agent, and optionally a pharmaceutically acceptable excipient, carrier or diluent. In various embodiments, a pharmaceutical composition comprises a therapeutically effective amount of an agent that inhibits the glutamate/cystine transporter x_{c}⁻, and optionally a pharmaceutically acceptable excipient, carrier or diluent. Optionally, the two agents may be in the same pharmaceutical composition. In one embodiment, a pharmaceutical composition encompasses a composition comprising the active ingredient(s), and the inert ingredient(s) that make up the carrier, as well as any product that results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the disclosure and a pharmaceutically acceptable excipient, carrier or diluent.

“Pharmaceutically acceptable carrier” refers to any of the standard pharmaceutical carriers, buffers, and the like, such as a phosphate buffered saline solution, 5% aqueous solution of dextrose, and emulsions (e.g., an oil/water or water/oil emulsion). Non-limiting examples of excipients include adjuvants, binders, fillers, diluents, disintegrants, emulsifying agents, wetting agents, lubricants, glidants, sweetening agents, flavoring agents, and coloring agents. Suitable pharmaceutical carriers, excipients and diluents are described in Remington's Pharmaceutical Sciences, 19th Ed. (Mack Publishing Co., Easton, 1995). Preferred pharmaceutical carriers depend upon the intended mode of administration of the active agent. Typical modes of administration include enteral (e.g., oral) or parenteral (e.g., subcutaneous, intramuscular, intravenous or intraperitoneal injection; or topical, transdermal, or transmucosal administration).

A “pharmaceutically acceptable salt” is a salt that can be formulated into a compound for pharmaceutical use, including but not limited to metal salts (e.g., sodium, potassium, magnesium, calcium, etc.) and salts of ammonia or organic amines. Examples of cysteamine salts include hydrochloride, bitartrate and phosphocysteamine derivatives. Cystamine and cystamine salts derivatives include sulfated cystamine.

As used herein “pharmaceutically acceptable” or “pharmacologically acceptable” salt, ester or other derivative of an active agent comprise, for example, salts, esters or other derivatives refers to a material that is not biologically or otherwise undesirable, i.e., the material may be administered to an individual without causing any undesirable biological effects or without interacting in a deleterious manner with any of the components of the composition in which it is contained or with any components present on or in the body of the individual.

As used herein, the term “unit dosage form” refers to physically discrete units suitable as unitary dosages for human and animal subjects, each unit containing a predetermined quantity of a compound of the disclosure calculated in an amount sufficient to produce the desired effect, optionally in association with a pharmaceutically acceptable excipient, diluent, carrier or vehicle. The specifications for the novel unit dosage forms of the present disclosure depend on the particular compound employed and the effect to be achieved, and the pharmacodynamics associated with each compound in the host.

As used herein, the term “subject” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender. In various embodiments the subject is human.

### Ecitotoxicity Disorders

Excitotoxicity disorders result from excessive glutamate release in the central nervous system resulting in glutamate toxicity to the surrounding cells. Contemplated herein are methods of treating an excitotoxicity disorder using a cysteamine product in combination with an agent that inhibits the glutamate/cysteine antiporter x_{c}⁻. Exemplary excitotoxicity disorders contemplated herein include, but are not limited to, spinal cord injury, stroke or other ischemia, traumatic brain injury, chronic traumatic encephalopathy (CTE), hearing loss, neurodegenerative diseases, multiple sclerosis, Alzheimer's disease, amyotrophic lateral sclerosis (ALS), Parkinson's disease, Huntington's disease, concussion, and CNS depressant-withdrawal syndrome.

### Huntington's Disease

Huntington's disease (HD) is an adult-onset neurodegenerative disorder for which treatment strategies have helped address certain symptoms of HD, but remain ineffective at truly treating the disease. HD is an autosomal dominant genetic disorder with a prevalence of about 5-10 per 100,000 in the Caucasian population. Clinical symptoms include chorea and behavioral disorders but the most problematic features of the disease are slowly progressive motor dysfunction and impaired cognition (Ha et al., Curr Opin Neurol 25(4):491-8, 2012). The pathology of HD is characterized by the presence of neuritic and intranuclear inclusions in neurons and relatively selective neural loss in the striatum and the deeper layers of the cerebral cortex. HD is caused by a Cytosine-Adenine-Guanine (CAG) triplet repeat expansion in the first exon of the HTT gene leading to an expanded polyglutamine stretch in the huntingtin protein (The Huntington's Disease Collaborative Research Group. A novel gene containing a trinucleotide repeat that is expanded and unstable on Huntington's disease chromosomes. Cell 72(6):971-83, 1993). HD develops when the polyglutamine expansion exceeds 35 CAG, a point that enlarges the polyglutamine stretch past a critical threshold that predisposes to aggregation. There is an inverse correlation between the number of CAG and the age at onset (Andrew et al., Nat Genet 4(4):398-403, 1993). Mutant huntingtin has been implicated in the disruption of many cellular processes, including protein clearance, protein-protein interaction, mitochondrial function, axonal trafficking, N-methyl-D-aspartate receptor activation, gene transcription and post-translational modification (Zuccato et al., Physiol Rev 2010; 90(3):905-81, Labbadia et al., Trends Biochem Sci 2013; 38(8):378-85). Although mutant huntingtin has a widespread distribution in neuronal and non-neuronal tissues, the medium spiny GABAergic neurons of the striatum exhibit the most pronounced vulnerability (Labbadia et al., Trends Biochem Sci 2013; 38(8):378-85).

Huntington's Disease is often defined or characterized by onset of symptoms and progression of decline in motor and neurological function. HD can be broken into five stages: Patients with early HD (stages 1 and 2) have increasing concerns about cognitive issues, and these concerns remain constant during moderate/intermediate HD (stages 3 and 4). Patients with late-stage or advanced HD (stage 5) have a lack of cognitive ability (Ho et al., Clin Genet. September 2011; 80(3):235-239).

Progression of the stages can be observed as follows: Early Stage (stage 1), in which the person is diagnosed as having HD and can function fully both at home and work. Early Intermediate Stage (stage 2), the person remains employable but at a lower capacity and are able to manage their daily affairs with some difficulties. Late Intermediate Stage (stage 3), the person can no longer work and/or manage household responsibilities and. need help or supervision to handle daily financial and other daily affairs. Early Advanced Stage patients (stage 4) are no longer independent in daily activities but is still able to live at home supported by their family or professional careers. In the Advanced Stage (stage 5), the person requires complete support in daily activities and professional nursing care is usually needed. Patients with HD usually die about 15 to 20 years after their symptoms first appear.

In intermediate stages, as the disease progresses, the initial motor symptoms will gradually develop into more obvious involuntary movements such as jerking and twitching of the head, neck, arms and legs. These movements may interfere with walking, speaking and swallowing. People at this stage of Huntington's often look as if they're drunk: they stagger when they walk and their speech is slurred. They have increasing difficulty working or managing a household, but can still deal with most activities of daily living. The advanced stages of HD typically involve fewer involuntary movements and more rigidity. Patients in these stages of HD can no longer manage the activities of daily living. Difficulties with swallowing, communication and weight loss are common in the advanced stage.

Chorea is the most common movement disorder seen in HD. Initially, mild chorea resembles fidgetiness. As the disease progresses, chorea gradually moves towards and is replaced by dystonia and parkinsonian features, such as bradykinesia, rigidity, and postural instability. In advanced disease, patients develop an akinetic-rigid syndrome, with minimal or no chorea, as well as spasticity, clonus, and extensor plantar responses. Dysarthria and dysphagia are common. Abnormal eye movements, tics and myoclonus may be seen in patients with HD. Juvenile HD (Westphal variant), defined as having an age of onset of younger than 20 years, is characterized by parkinsonian features, dystonia, long-tract signs, dementia, epilepsy, and mild or even absent chorea.

Cognitive decline is also characteristic of HD, and the rate of progression can vary among individual patients. Dementia and the psychiatric features of HD are often the earliest of functional impairment. Dementia syndrome associated with HD includes early onset behavioral changes, such as irritability, untidiness, and loss of interest, followed by slowing of cognition, impairment of intellectual function, and memory disturbances. This pattern corresponds well to the syndrome of subcortical dementia, and it has been suggested to reflect dysfunction of frontal-subcortical neuronal circuitry.

Early stages of HD are characterized by deficits in short-term memory, followed by motor dysfunction and a variety of cognitive changes in the intermediate stages of dementia (Loy et al., PLoS Curr. 2013; 5: Cleret de Langavant et al., PLoS One. 2013; 8(4):e61676). These deficits include diminished verbal fluency, problems with attention, executive function, visuospatial processing, and abstract reasoning. Language skills become affected in the final stages of the illness, resulting in marked word-retrieval deficiency.

HD can also manifest in behavioral disorders, including depression, with a small percentage of patients experiencing bouts of mania characteristic of bipolar disorder, an increased rate of suicide, and psychosis, obsessive-compulsive symptoms, sexual and sleep disorders, and changes in personality.

### Parkinson's Disease

Parkinson's disease (PD) is a complex neurodegenerative disorder involving the predominant loss of dopaminergic neurons in the substantia nigra pars compacta (SNc), subsequent decay of the nigrostriatal tract and associated movement anomalies such as rigidity, bradykinesia and tremor. Pathological features associated with substantial nigra degeneration include mitochondrial abnormalities, loss of antioxidant enzyme systems and reduced glutathione (GSH) levels (Bharath et al., Biochem Pharmacol. 64:1037-48, 2002).

Stages of a Parkinson's disease patient is described by Hoehn and Yahr in following five distinct stages depending on the symptoms (Hoehn M M, Yahr M D, Parkinsonism: onset, progression and mortality. Neurology 1967, 17:427-42). Stage I: (mild or early disease): symptoms affect only one side of the body. Stage II: both sides of the body are affected, but posture remains normal. Stage III: (moderate disease): both sides of the body are affected, and there is mild imbalance during standing or walking, however, the person remains independent. Stage IV: (advanced disease): both sides of the body are affected, and there is disabling instability while standing or walking. The person in this stage requires substantial help. Stage V: severe, fully developed disease is present. The person is restricted to a bed or chair.

### Ischemia

Ischemia refers to a condition resulting from a decrease or lack of blood flow and oxygen to a part of the body such as the brain, heart, or other tissue. Ischemic injury refers generally to the damage to a tissue that is distal or otherwise effected by the loss of blood flow and oxygen. Ischemic injury is often a result of the lack of oxygen and fluids, but also includes inflammatory cascades. For example, ischemia and ischemic injury can occur as a result of cardiac, pulmonary or brain injury, organ transplantation or surgical procedure, or a disease or disorder.

Acute ischemia is most often recognized in strokes and cardiac damage. However, there are a number of disorders and injuries that cause ischemic events leading to cell death and tissue damage. Strokes, cerebrovascular events and cardio vascular events are the result of an acute obstruction of cerebral or cardiac blood flow to a region of the brain or heart, respectively. There are approximately 500,000 cases of stroke each year in the United States, of which 30% are fatal, and hence stroke is the third leading cause of death in the United States. Approximately 80% of strokes are “ischemic” and result from an acute occlusion of a cerebral artery with resultant reduction in blood flow. The remainder are “hemorrhagic”, which are due to rupture of a cerebral artery with hemorrhage into brain tissue and consequent obstruction of blood flow due to lack of flow in the distal region of the ruptured vessel and local tissue compression, creating ischemia.

Stroke commonly affects individuals older than 65 years. In 1996, the FDA approved the use of tissue plasminogen activator (tPA) as therapy for acute ischemic stroke, based on a limited number of controlled trials. Approximately twenty percent of strokes may involve bleeding within the brain, which damages nearby brain tissue (for example, a hemorrhagic stroke). Hemorrhagic stroke occurs when a blood vessel bursts inside the brain. The brain is sensitive to bleeding and damage can occur rapidly, either because of the presence of the blood itself, or because the fluid increases pressure on the brain and harms it by pressing it against the skull. The surrounding tissues of the brain resist the expansion of the bleeding, which is finally contained by forming a mass (for example, an intracerebral hematoma). Both swelling and hematoma will compress and displace normal brain tissue.

There appears to be a correlation between an early reduction in glutathione levels in ischemia and the activation of lipooxygenases by the inflammatory cascade, which may play a role in ischemia-induced nerve cell loss. In vitro cell culture assays have shown that inhibitors of lipoxygenase 12-LOX block glutamate-induced cell death, and both 5- and 12-LOX inhibitors block ischemic injury in hippocampal slice cultures.

### Alzheimer's Disease

Alzheimer's disease (AD) is characterized by chronic, progressive neurodegeneration. Neurodegeneration in AD involves early synaptotoxicity, neurotransmitter disturbances, accumulation of extracellular β-amyloid (Aβ) deposits and intracellular neurofibrils, and gliosis and at later stages loss of neurons and associated brain atrophy (Danysz et al., Br J Pharmacol. 167:324-352, 2012). Early studies indicated Aβ peptides may have the ability to enhance glutamate toxicity in human cerebral cortical cell cultures (Mattson et al., J Neurosci. 12:376-389, 1992; Li et al., J Neurosci. 31(18):6627-38, 2011).

It is contemplated herein that administration of a cysteamine product or composition as described herein in combination with an agent that inhibits the glutamate/cysteine antiporter x_{c}⁻ can alleviate or treat one or more symptoms associated with excitotoxicity disease or disorder. Such symptoms, include but are not limited to, one or more motor skills, cognitive function, dystonia, chorea, psychiatric symptoms such as depression, brain and striatal atrophies, and neuronal dysfunction.

It is contemplated that the administration results in a slower progression of total motor score compared to a subject not receiving cysteamine composition and x_{c}⁻ inhibitor as described herein. In some embodiments, the slower progression is a result in improvement in one or more motor scores selected from the group consisting of chorea subscore, balance and gait subscore, hand movements subscore, eye movement subscore, maximal dystonia subscore and bradykinesia assessment.

Additional indicia of a slower decline in symptoms of HD are measured using change from baseline in one or more of the following parameters: using standardized tests for (i) functional assessment (e.g., UHDRS Total Functional Capacity, LPAS, Independence Scale); (ii) neuropsychological assessment (e.g., UHDRS Cognitive Assessment, Mattis Dementia Rating Scale, Trail Making Test A and B, Figure Cancellation Test, Hopkins Verbal Learning Test, Articulation Speed Test); (iii) psychiatric assessment (UHDRS Behavioral Assessment, Montgomery and Asberg Depression Rating Scale) and (iv) cognitive assessment (e.g., Dementia Outcomes Measurement Suite (DOMS)).

In certain embodiments, alteration in one or more symptoms in patients receiving cysteamine composition in combination with an agent that inhibits the glutamate/cysteine antiporter x_{c}⁻ is shown to be beneficial by at least 10%, 20%, 25%, 30%, 35%, 40%, 45%, 50%, 55%, 60%, 65%, 70%, 75% or more compared to baseline assessment of the symptom. In certain embodiments, the rate of progression or decline in total motor score is slowed, by at least 20%, 25%, 30%, 35%, 40%, 45%, 50%, 55%, 60%, 65%, 70%, 75% or more. Measurement may be performed using techniques known in the art, e.g., the Unified Huntington Disease Rating Scale (UHDRS), Bradykinesia Ratings Scale, and Lindop Parkinson's Assessment Scale (LPAS).

In certain embodiments, the symptoms are measured at 3 months, 6 months, 12 months, 18 months or 2 years or more after administration.

The disclosure also provides a method for slowing the progression of brain and striatal atrophies and/or treating dystonia in a subject suffering from an excitotoxicity disease comprising administering to a subject in need thereof a cysteamine composition in combination with an agent that inhibits the glutamate/cysteine antiporter x_{c}⁻.

It is contemplated that the methods described herein and effects observed with a cysteamine composition are also carried out and observed after administration of a diffusible small thiol compound as described herein.

### Cysteamine/Cystamine

Cysteamine (HS—CH₂—CH₂—NH₂) is a small sulfhydryl compound that is able to cross cell membranes easily due to its small size. Cysteamine plays a role in formation of the tripeptide glutathione (GSH), and is currently FDA approved for use in the treatment of cystinosis, an intra-lysosomal cystine storage disorder. In cystinosis, cysteamine acts by converting cystine to cysteine and cysteine-cysteamine mixed disulfide, which are then both able to leave the lysosome through the cysteine and lysine transporters respectively (Gahl et al., N Engl J Med 2002; 347(2):111-21). Within the cytosol the mixed disulfide can be reduced by its reaction with glutathione and the cysteine released can be used for further GSH synthesis. Treatment with cysteamine has been shown to result in lowering of intracellular cystine levels in circulating leukocytes (Dohil et al., J. Pediatr 148(6):764-9, 2006). The synthesis of GSH from cysteine is catalyzed by two enzymes, gamma-glutamylcysteine synthetase and GSH synthetase. This pathway occurs in almost all cell types, with the liver being the major producer and exporter of GSH. The reduced cysteine-cysteamine mixed disulfide will also release cysteamine, which, in theory is then able to re-enter the lysosome, bind more cystine and repeat the process (Dohil et al., J Pediatr 2006; 148(6):764-9). In a study in children with cystinosis, enteral administration of cysteamine resulted in increased plasma cysteamine levels, which subsequently caused prolonged efficacy in the lowering of leukocyte cystine levels (Dohil et al., J Pediatr 2006; 148(6):764-9). This may have been due to “re-cycling” of cysteamine when adequate amounts of drug reached the lysosome. If cysteamine acts in this fashion, then GSH production may also be significantly enhanced.

In addition, sulfhydryl (SH) compounds such as cysteamine, cystamine, and glutathione are active intracellular antioxidants. Cysteamine protects animals against bone marrow and gastrointestinal radiation syndromes. The rationale for the important anti-oxidant properties of SH compounds is further supported by observations in mitotic cells. These are the most sensitive to radiation injury in terms of cell reproductive death and are noted to have the lowest level of SH compounds. Conversely, S-phase cells, which are the most resistant to radiation injury using the same criteria, have demonstrated the highest levels of inherent SH compounds. In addition, when mitotic cells were treated with cysteamine, they became very resistant to radiation. It has also been noted that cysteamine may directly protect cells against induced mutations. The protection is thought to result from scavenging of free radicals, either directly or via release of protein-bound GSH. An enzyme that liberates cysteamine from coenzyme A has been reported in avian liver and hog kidney. Recently, studies have reported a protective effect of cysteamine against the hepatotoxic agents acetaminophen, bromobenzene, and phalloidine.

Cystamine, in addition to its role as a radioprotectant, has been found to alleviate tremors and prolong life in mice with the gene mutation for Huntington's disease (HD). The drug may work by increasing the activity of proteins that protect nerve cells, or neurons, from degeneration. However, due to the current methods and formulation of delivery of cystamine, degradation and poor uptake require excessive dosing.

Cysteamine is also discussed in (Prescott et al., Lancet 1972; 2(7778):652; Prescott et al., Br Med J 1978; 1(6116):856-7; Mitchell et al., Clin Pharmacol Ther 1974; 16(4):676-84; Toxicol Appl Pharmacol. 1979 48(2):221-8; Qiu et al., World J Gastroenterol. 13:4328-32, 2007. Unfortunately, the sustained concentrations of cysteamine necessary for therapeutic effect are difficult to maintain due to rapid metabolism and clearance of cysteamine from the body, with nearly all administered cysteamine converted to taurine in a matter of hours. These difficulties are transferred to patients in the form of high dosing levels and frequencies, with all of the consequent unpleasant side effects associated with cysteamine (e.g., gastrointestinal distress and body odor) See the package insert for CYSTAGON® (cysteamine bitartrate). International Publication No. WO 2007/089670 discloses enterically coated cysteamine products and a method of reducing dosing frequency of cysteamine. Cysteamine is also addressed in International Patent Application Nos. WO 2009/070781, and WO 2007/089670, and U.S. Patent Publication Nos. 20110070272, 20090048154, and 20050245433.

### Cysteamine Compositions

In another aspect, the disclosure provides cysteamine compositions for use in the methods described herein.

A “cysteamine composition” in the present disclosure refers generally to cysteamine or a pharmaceutically acceptable salt thereof or cystamine or a pharmaceutically acceptable salt thereof, including a biologically active metabolite or derivative thereof, structural analogs thereof, or combination of cysteamine and cystamine, and includes cysteamine or cystamine salts, esters, amides, alkylate compounds, prodrugs, analogs, phosphorylated compounds, sulfated compounds, nitrosylated and glycosylated compounds or other chemically modified forms thereof (e.g., chemically modified forms prepared by labeling with radionucleotides or enzymes and chemically modified forms prepared by attachment of polymers such as polyethylene glycol). Thus, the cysteamine or cystamine can be administered in the form of a pharmacologically acceptable salt, ester, amide, prodrug or analog or as a combination thereof. In various embodiments, the cysteamine product includes cysteamine, cystamine or derivatives thereof. In any of the embodiments described herein, a cysteamine product may optionally exclude N-acetylcysteine.

Salts, esters, amides, prodrugs and analogs of the active agents may be prepared using standard procedures known to those skilled in the art of synthetic organic chemistry and described, for example, by J. March, “Advanced Organic Chemistry: Reactions, Mechanisms and Structure,” 4th Ed. (New York: Wiley-Interscience, 1992). For example, basic addition salts are prepared from the neutral drug using conventional means, involving reaction of one or more of the active agent's free hydroxyl groups with a suitable base. Generally, the neutral form of the drug is dissolved in a polar organic solvent such as methanol or ethanol and the base is added thereto. The resulting salt either precipitates or may be brought out of solution by addition of a less polar solvent. Suitable bases for forming basic addition salts include, but are not limited to, inorganic bases such as sodium hydroxide, potassium hydroxide, ammonium hydroxide, calcium hydroxide, trimethylamine, or the like. Preparation of esters involves functionalization of hydroxyl groups which may be present within the molecular structure of the drug. The esters are typically acyl-substituted derivatives of free alcohol groups, i.e., moieties which are derived from carboxylic acids of the formula R—COOH where R is alkyl, and typically is lower alkyl. Esters can be reconverted to the free acids, if desired, by using conventional hydrogenolysis or hydrolysis procedures. Preparation of amides and prodrugs can be carried out in an analogous manner. Other derivatives and analogs of the active agents may be prepared using standard techniques known to those skilled in the art of synthetic organic chemistry, or may be deduced by reference to the pertinent literature.

### Pharmaceutical Formulations

The disclosure provides for use of cysteamine products and agents that inhibit the x_{c}⁻ transporter in the treatment of excitotoxicity diseases or disorders, such as Huntington's Disease, Parkinson's disease, ischemia, or Alzheimer's disease (e.g., to slow or improve motor skills, cognitive function and promote neuronal regeneration). To administer cysteamine products and/or an agent that inhibits x_{c}⁻ to patients or test animals, it is preferable to formulate the therapeutics in a composition comprising one or more pharmaceutically acceptable carriers. Pharmaceutically or pharmacologically acceptable carriers or vehicles refer to molecular entities and compositions that do not produce allergic, or other adverse reactions when administered using routes well-known in the art, as described below, or are approved by the U.S. Food and Drug Administration or a counterpart foreign regulatory authority as an acceptable additive to orally or parenterally administered pharmaceuticals. Pharmaceutically acceptable carriers include any and all clinically useful solvents, dispersion media, coatings, antibacterial and antifungal agents, isotonic and absorption delaying agents and the like.

Pharmaceutical carriers include pharmaceutically acceptable salts, particularly where a basic or acidic group is present in a compound. For example, when an acidic substituent, such as —COOH, is present, the ammonium, sodium, potassium, calcium and the like salts, are contemplated for administration. Additionally, where an acid group is present, pharmaceutically acceptable esters of the compound (e.g., methyl, tert-butyl, pivaloyloxymethyl, succinyl, and the like) are contemplated as preferred forms of the compounds, such esters being known in the art for modifying solubility and/or hydrolysis characteristics for use as sustained release or prodrug formulations.

When a basic group (such as amino or a basic heteroaryl radical, such as pyridyl) is present, then an acidic salt, such as hydrochloride, hydrobromide, acetate, maleate, pamoate, phosphate, methanesulfonate, p-toluenesulfonate, and the like, is contemplated as a form for administration.

In addition, compounds may form solvates with water or common organic solvents. Such solvates are contemplated as well.

The cysteamine products or agent that inhibits x_{c}⁻ may be administered orally, parenterally, transocularly, intranasally, transdermally, transmucosally, by inhalation spray, vaginally, rectally, or by intracranial injection. The term parenteral as used herein includes subcutaneous injections, intravenous, intramuscular, intracisternal injection, or infusion techniques. Administration by intravenous, intradermal, intramusclar, intramammary, intraperitoneal, intrathecal, retrobulbar, intrapulmonary injection and or surgical implantation at a particular site is contemplated as well. Generally, compositions for administration by any of the above methods are essentially free of pyrogens, as well as other impurities that could be harmful to the recipient. Further, compositions for administration parenterally are sterile.

Pharmaceutical compositions of the disclosure containing a cysteamine product, e.g., cyteamine bitartrate, or an agent that inhibits x_{c}⁻ as an active ingredient may contain pharmaceutically acceptable carriers or additives depending on the route of administration. Examples of such carriers or additives include water, a pharmaceutically acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the disclosure.

Formulation of the pharmaceutical composition will vary according to the route of administration selected (e.g., solution, emulsion). An appropriate composition comprising the cysteamine product to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers.

A variety of aqueous carriers, e.g., water, buffered water, 0.4% saline, 0.3% glycine, or aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

In some embodiments, the cysteamine product or an agent that inhibits x_{c}⁻ disclosed herein can be lyophilized for storage and reconstituted in a suitable carrier prior to use. Any suitable lyophilization and reconstitution techniques can be employed. It is appreciated by those skilled in the art that lyophilization and reconstitution can lead to varying degrees of activity loss and that use levels may have to be adjusted to compensate.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

In one embodiment, the disclosure provides use of an enterically coated cysteamine product composition, e.g., cysteamine bitartrate. Enteric coatings prolong release until the cysteamine product reaches the intestinal tract, typically the small intestine. Because of the enteric coatings, delivery to the small intestine is improved thereby improving uptake of the active ingredient while reducing gastric side effects. Exemplary enterically coated cysteamine products are described in International Publication No. WO 2007/089670 and in International Patent Applications PCT/US14/42607 and PCT/US14/42616.

In some embodiments, the coating material is selected such that the therapeutically active agent is released when the dosage form reaches the small intestine or a region in which the pH is greater than pH 4.5. In various embodiments, the formulation releases at a pH of about 4.5 to 6.5, 4.5 to 5.5, 5.5 to 6.5 or about pH 4.5, 5.0, 5.5, 6.0 or 6.5.

The coating may be a pH-sensitive materials, which remain intact in the lower pH environs of the stomach, but which disintegrate or dissolve at the pH commonly found in the small intestine of the patient. For example, the enteric coating material begins to dissolve in an aqueous solution at pH between about 4.5 to about 5.5. For example, pH-sensitive materials will not undergo significant dissolution until the dosage form has emptied from the stomach. The pH of the small intestine gradually increases from about 4.5 to about 6.5 in the duodenal bulb to about 7.2 in the distal portions of the small intestine. In order to provide predictable dissolution corresponding to the small intestine transit time of about 3 hours (e.g., 2-3 hours) and permit reproducible release therein, the coating should begin to dissolve at the pH range within the small intestine. Therefore, the amount of enteric polymer coating should be sufficient to substantially dissolved during the approximate three hour transit time within the small intestine, such as the proximal and mid-intestine.

Enteric coatings have been used for many years to arrest the release of the drug from orally ingestible dosage forms. Depending upon the composition and/or thickness, the enteric coatings are resistant to stomach acid for required periods of time before they begin to disintegrate and permit release of the drug in the lower stomach or upper part of the small intestines. Examples of some enteric coatings are disclosed in U.S. Pat. No. 5,225,202 which is incorporated by reference fully herein. As set forth in U.S. Pat. No. 5,225,202, some examples of coating previously employed are beeswax and glyceryl monostearate; beeswax, shellac and cellulose; and cetyl alcohol, mastic and shellac, as well as shellac and stearic acid (U.S. Pat. No. 2,809,918); polyvinyl acetate and ethyl cellulose (U.S. Pat. No. 3,835,221); and neutral copolymer of polymethacrylic acid esters (Eudragit L30D) (F. W. Goodhart et al., Pharm. Tech., pp. 64-71, April 1984); copolymers of methacrylic acid and methacrylic acid methylester (Eudragits), or a neutral copolymer of polymethacrylic acid esters containing metallic stearates (Mehta et al., U.S. Pat. Nos. 4,728,512 and 4,794,001). Such coatings comprise mixtures of fats and fatty acids, shellac and shellac derivatives and the cellulose acid phthlates, e.g., those having a free carboxyl content. See, Remington's at page 1590, and Zeitova et al. (U.S. Pat. No. 4,432,966), for descriptions of suitable enteric coating compositions. Accordingly, increased adsorption in the small intestine due to enteric coatings of cysteamine product compositions can result in improved efficacy.

Generally, the enteric coating comprises a polymeric material that prevents cysteamine product release in the low pH environment of the stomach but that ionizes at a slightly higher pH, typically a pH of 4 or 5, and thus dissolves sufficiently in the small intestines to gradually release the active agent therein. Accordingly, among the most effective enteric coating materials are polyacids having a pKa in the range of about 3 to 5. Suitable enteric coating materials include, but are not limited to, polymerized gelatin, shellac, methacrylic acid copolymer type CNF, cellulose butyrate phthalate, cellulose hydrogen phthalate, cellulose proprionate phthalate, polyvinyl acetate phthalate (PVAP), cellulose acetate phthalate (CAP), cellulose acetate trimellitate (CAT), hydroxypropyl methylcellulose phthalate, hydroxypropyl methylcellulose acetate, dioxypropyl methylcellulose succinate, carboxymethyl ethylcellulose (CMEC), hydroxypropyl methylcellulose acetate succinate (HPMCAS), and acrylic acid polymers and copolymers, typically formed from methyl acrylate, ethyl acrylate, methyl methacrylate and/or ethyl methacrylate with copolymers of acrylic and methacrylic acid esters (Eudragit NE, Eudragit RL, Eudragit RS). In one embodiment, the cysteamine product composition is administered in an oral delivery vehicle, including but not limited to, tablet or capsule form. Tablets are manufactured by first enterically coating the cysteamine product. A method for forming tablets herein is by direct compression of the powders containing the enterically coated cysteamine product, optionally in combination with diluents, binders, lubricants, disintegrants, colorants, stabilizers or the like. As an alternative to direct compression, compressed tablets can be prepared using wet-granulation or dry-granulation processes. Tablets may also be molded rather than compressed, starting with a moist material containing a suitable water-soluble lubricant.

The preparation of delayed, controlled or sustained/extended release forms of pharmaceutical compositions with the desired pharmacokinetic characteristics is known in the art and can be accomplished by a variety of methods. For example, oral controlled delivery systems include dissolution-controlled release (e.g., encapsulation dissolution control or matrix dissolution control), diffusion-controlled release (reservoir devices or matrix devices), ion exchange resins, osmotic controlled release or gastroretentive systems. Dissolution controlled release can be obtained, e.g., by slowing the dissolution rate of a drug in the gastrointestinal tract, incorporating the drug in an in soluble polymer, and coating drug particles or granules with polymeric materials of varying thickness. Diffusion controlled release can be obtained, e.g., by controlling diffusion through a polymeric membrane or a polymeric matrix. Osmotically controlled release can be obtained, e.g., by controlling solvent influx across a semipermeable membrane, which in turn carries the drug outside through a laser-drilled orifice. The osmotic and hydrostatic pressure differences on either side of the membrane govern fluid transport. Prolonged gastric retention may be achieved by, e.g., altering density of the formulations, bioadhesion to the stomach lining, or increasing floating time in the stomach. For further detail, see the Handbook of Pharmaceutical Controlled Release Technology, Wise, ed., Marcel Dekker, Inc., New York, N.Y. (2000), incorporated by reference herein in its entirety, e.g. Chapter 22 (“An Overview of Controlled Release Systems”).

The concentration of cysteamine product in these formulations can vary widely, for example from less than about 0.5%, usually at or at least about 1% to as much as 15 or 20% by weight and are selected primarily based on fluid volumes, manufacturing characteristics, viscosities, etc., in accordance with the particular mode of administration selected. Actual methods for preparing administrable compositions are known or apparent to those skilled in the art and are described in more detail in, for example, Remington's Pharmaceutical Science, 15th ed., Mack Publishing Company, Easton, Pa. (1980) and further editions thereof.

Compositions useful for administration may be formulated with uptake or absorption enhancers to increase their efficacy. Such enhancers include, for example, salicylate, glycocholate/linoleate, glycholate, aprotinin, bacitracin, SDS, caprate and the like. See, e.g., Fix (J. Pharm. Sci., 85:1282-1285, 1996) and Oliyai and Stella (Ann. Rev. Pharmacol. Toxicol., 32:521-544, 1993).

The enterically coated cysteamine product can comprise various excipients, as is well known in the pharmaceutical art, provided such excipients do not exhibit a destabilizing effect on any components in the composition. Thus, excipients such as binders, bulking agents, diluents, disintegrants, lubricants, fillers, carriers, and the like can be combined with the cysteamine product. Oral delivery vehicles contemplated for use herein include tablets, capsules, comprising the product. For solid compositions, diluents are typically necessary to increase the bulk of a tablet or capsule so that a practical size is provided for compression. Suitable diluents include dicalcium phosphate, calcium sulfate, lactose, cellulose, kaolin, mannitol, sodium chloride, dry starch and powdered sugar. Binders are used to impart cohesive qualities to an oral delivery vehicle formulation, and thus ensure that a tablet remains intact after compression. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, hydroxyethyl cellulose, hypromellose, and the like), and Veegum. Lubricants are used to facilitate oral delivery vehicle manufacture; examples of suitable lubricants include, for example, magnesium stearate, calcium stearate, and stearic acid, and are typically present at no more than approximately 1 weight percent relative to tablet weight. Disintegrants are used to facilitate oral delivery vehicle, (e.g., a tablet) disintegration or “breakup” after administration, and are generally starches, clays, celluloses, algins, gums or crosslinked polymers. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. If desired, flavoring, coloring and/or sweetening agents may be added as well. Other optional components for incorporation into an oral formulation herein include, but are not limited to, preservatives, suspending agents, thickening agents, and the like. Fillers include, for example, insoluble materials such as silicon dioxide, titanium oxide, alumina, talc, kaolin, powdered cellulose, microcrystalline cellulose, and the like, as well as soluble materials such as mannitol, urea, sucrose, lactose, dextrose, sodium chloride, sorbitol, and the like.

A pharmaceutical composition may also comprise a stabilizing agent such as hydroxypropyl methylcellulose or polyvinylpyrrolidone, as disclosed in U.S. Pat. No. 4,301,146. Other stabilizing agents include, but are not limited to, cellulosic polymers such as hydroxypropyl cellulose, hydroxyethyl cellulose, methyl cellulose, ethyl cellulose, cellulose acetate, cellulose acetate phthalate, cellulose acetate trimellitate, hydroxypropyl methylcellulose phthalate, microcrystalline cellulose and carboxymethylcellulose sodium; and vinyl polymers and copolymers such as polyvinyl acetate, polyvinylacetate phthalate, vinylacetate crotonic acid copolymer, and ethylene-vinyl acetate copolymers. The stabilizing agent is present in an amount effective to provide the desired stabilizing effect; generally, this means that the ratio of cysteamine product to the stabilizing agent is at least about 1:500 w/w, more commonly about 1:99 w/w.

The tablet, capsule, or other oral delivery system is manufactured by enterically coating the cysteamine product. A method for forming tablets herein is by direct compression of the powders containing the enterically coated cysteamine product, optionally in combination with diluents, binders, lubricants, disintegrants, colorants, stabilizers or the like. As an alternative to direct compression, compressed tablets can be prepared using wet-granulation or dry-granulation processes. Tablets may also be molded rather than compressed, starting with a moist material containing a suitable water-soluble lubricant.

In various embodiments, the enterically coated cysteamine product is granulated and the granulation is compressed into a tablet or filled into a capsule. In certain embodiments, the granules are enterically coated prior to compressing into a tablet or capsule. Capsule materials may be either hard or soft, and are typically sealed, such as with gelatin bands or the like. Tablets and capsules for oral use will generally include one or more commonly used excipients as discussed herein.

In a further embodiment, the cystemine product is formulated as a capsule. In one embodiment, the capsule comprises the cysteamine product and the capsule is then enterically coated. Capsule formulations are prepared using techniques known in the art.

A suitable pH-sensitive polymer is one which will dissolve in intestinal environment at a higher pH level (pH greater than 4.5), such as within the small intestine and therefore permit release of the pharmacologically active substance in the regions of the small intestine and not in the upper portion of the GI tract, such as the stomach.

In various embodiments, exemplary cysteamine or cystamine product formulations contemplated for use in the present methods are described in International Patent Applications PCT/US14/42607 and PCT/US14/42616.

For administration of the dosage form, i.e., the tablet or capsule comprising the enterically coated cysteamine product, a total weight in the range of approximately 100 mg to 1000 mg is used. In various embodiments, the tablet or capsule comprises 25, 50, 75, 100, 125, 150, 175, 200, 225, 250, 275, 300, 400 or 500 mg active ingredient, and multiple tablets or capsules are administered to reach the desired dosage. The dosage form is orally administered to a subject need thereof.

In addition, various prodrugs can be “activated” by use of the enterically coated cysteamine. Prodrugs are pharmacologically inert, they themselves do not work in the body, but once they have been absorbed, the prodrug decomposes. The prodrug approach has been used successfully in a number of therapeutic areas including antibiotics, antihistamines and ulcer treatments. The advantage of using prodrugs is that the active agent is chemically camouflaged and no active agent is released until the drug has passed out of the gut and into the cells of the body. For example, a number of produgs use S—S bonds. Weak reducing agents, such as cysteamine, reduce these bonds and release the drug. Accordingly, the compositions of the disclosure are useful in combination with pro-drugs for timed release of the drug. In this aspect, a pro-drug can be administered followed by administration of an enterically coated cysteamine compositions of the disclosure (at a desired time) to activate the pro-drug.

Prodrugs of cysteamine have been described previously. See, e.g., Andersen et al., In Vitro Evaluation of Novel Cysteamine Prodrugs Targeted to g-Glutamyl Transpeptidase (poster presentation), which describes S-pivaloyl cysteamine derivatives, S-benzoyl cysteamine derivatives, S-acetyl cysteamine derivatives and S-benzoyl cysteamine)glutamate-ethyl ester). Omran et al., Bioorg Med Chem Lett. 2011 Apr. 15; 21(8):2502-4 describes a folate pro-drug of cystamine as a treatment for nephropathic cystinosis.

Thiazolidine prodrugs are also contemplated, and can be made as described previously. See e.g., Wilmore et al., J. Med. Chem., 44 (16):2661-2666, 2001 and Cardwell, Wash., “Synthesis And Evaluation Of Novel Cysteamine Prodrugs” 2006, Thesis, Univ. of Sunderland.

Pharmaceutical compositions comprising a diffusible small thiol compound, e.g., thiomandelic acid, Captopril, Thiorphan, N-acetylcysteine, 2,3-dimercaptosuccinate, 2,3-dimercaprol, penicillamine, glutathione, cysteine, homocysteine, Zofenoprilat, Tiopronin, pantetheine, coenzyme A, amifostine, WR-1065, thiophenol, thioacetic acid, 2-mercaptoethanol, dithiothreitol, dithioerythritol, 2-mercaptoindole, and disulfides, mixed or symmetrical, of any of the above, for use in the methods are also contemplated.

### Dosing and Administration

The cysteamine product and/or an agent that inhibits x_{c}⁻ are each administered in a therapeutically effective amount; typically, in unit dosage form. The amount of product administered is, of course, dependent on the age, weight, and general condition of the patient, the severity of the condition being treated, and the judgment of the prescribing-physician. Suitable therapeutic amounts will be known to those skilled in the art and/or are described in the pertinent reference texts and literature. Current non-enterically coated doses of cysteamine are about 1.35 g/m² body surface area and are administered 4-5 times per day (Levtchenko et al., Pediatr Nephrol. 21:110-113, 2006). In one aspect, the dose of therapeutic is administered either one time per day or multiple times per day.

The cysteamine product may be administered less than four time per day, e.g., one, two or three times per day. In various embodiments, the total daily dose of cysteamine or a pharmaceutically acceptable salt thereof or cystamine or a pharmaceutically acceptable salt thereof for treatment of an excitotoxicity disease or disorder described herein is between 200 to 1000, 500 to 2000 mg, 750 to 1750 mg, 1000 to 1500 mg, or may range between any two of the foregoing values. In various embodiments, the total daily dose of cysteamine product, e.g., cysteamine or a pharmaceutically acceptable salt thereof or cystamine or a pharmaceutically acceptable salt thereof, is 200, 300, 400, 500, 600, 700, 800, 900, 1000, 1100, 1200, 1300, 1400, 1500, 1600, 1700, 1800, 1900 or 2000 mg per day. It is contemplated that any of the foregoing doses is administered twice daily. It is further contemplated that any of the foregoing doses is administered in two equal doses daily. Optionally, the daily dose is administered in three doses.

In some embodiments, an effective dosage of cysteamine product may be within the range of 0.01 mg to 1000 mg per kg (mg/kg) of body weight per day. In some embodiments, the cysteamine, cystamine or pharmaceutically acceptable salt thereof is administered at a daily dose ranging from about 1 to about 50 mg/kg/day, or from about 10 mg/kg to about 250 mg/kg, or from about 100 mg/kg to about 250 mg/kg, or from about 60 mg/kg to about 100 mg/kg or from about 50 mg/kg to about 90 mg/kg, or from about 30 mg/kg to about 80 mg/kg, or from about 20 mg/kg to about 60 mg/kg, or from about 10 mg/kg to about 50 mg/kg, or from about 15 to about 25 mg/kg, or from about 15 to about 20 mg/kg or from about 10 to about 20 mg/kg. Further, the effective dose may be 0.5 mg/kg, 1 mg/kg, 5 mg/kg, 10 mg/kg, 15 mg/kg, 20 mg/kg/25 mg/kg, 30 mg/kg, 35 mg/kg, 40 mg/kg, 45 mg/kg, 50 mg/kg, 55 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, 225 mg/kg, 250 mg/kg, 275 mg/kg, 300 mg/kg, 325 mg/kg, 350 mg/kg, 375 mg/kg, 400 mg/kg, 425 mg/kg, 450 mg/kg, 475 mg/kg, 500 mg/kg, 525 mg/kg, 550 mg/kg, 575 mg/kg, 600 mg/kg, 625 mg/kg, 650 mg/kg, 675 mg/kg, 700 mg/kg, 725 mg/kg, 750 mg/kg, 775 mg/kg, 800 mg/kg, 825 mg/kg, 850 mg/kg, 875 mg/kg, 900 mg/kg, 925 mg/kg, 950 mg/kg, 975 mg/kg or 1000 mg/kg, or may range between any two of the foregoing values.

In some embodiments, the cysteamine product is administered at a total daily dose of from approximately 0.25 g/m² to 4.0 g/m² body surface area, e.g., at least about 0.3, 0.4, 0.5, 0.6, 0.7, 0.8, 0.9, 1.0, 1.1, 1.2, 1.3, 1.4, 1.5, 1.6, 1.7, 1.8, 1.9 or 2 g/m², or up to about 0.8, 0.9, 1.0, 1.1, 1.2, 1.3, 1.4, 1.5, 1.6, 1.7, 1.8, 1.9, 2.0, 2.2, 2.5, 2.7, 3.0, 3.25, 3.5 or 3.75 g/m² or may range between any two of the foregoing values. In some embodiments, the cysteamine product may be administered at a total daily dose of about 0.5-2.0 g/m² body surface area, or 1-1.5 g/m² body surface area, or 1-1.95 g/m² body surface area, or 0.5-1 g/m² body surface area, or about 0.7-0.8 g/m² body surface area, or about 1.35 g/m² body surface area, or about 1.3 to about 1.95 grams/m2/day, or about 0.5 to about 1.5 grams/m2/day, or about 0.5 to about 1.0 grams/m2/day, preferably at a frequency of fewer than four times per day, e.g. three, two or one times per day. Salts or esters of the same active ingredient may vary in molecular weight depending on the type and weight of the salt or ester moiety. For administration of enteric dosage form, e.g., a tablet or capsule or other oral dosage form comprising the enterically coated cysteamine product, a total weight in the range of approximately 100 mg to 1000 mg is used. In various embodiments, the tablet or capsule comprises 25, 50, 75, 100, 125, 150, 175, 200, 225, 250, 275, 300, 400 or 500 mg active ingredient, and multiple tablets or capsules are administered to reach the desired dosage

Inhibitors of x_{c}⁻ transport are also used at therapeutically effective amounts. Exemplary inhibitors of the x_{c}⁻ transporter include, but are not limited to, sulfasalazine, 4-s-carboxyphenylglycine, 4-s-sulfonylphenylglycine, sorafenib, erastin, and [(R,S)-4-[4′-carboxyphenyl]-phenylglycine.

Sulfasalazine is described in U.S. Pat. No. 7,498,047. Sulfasalazine has been shown to specifically inhibit the x_{c}⁻ transporter in dendritic cells and other cell types. Sulfasalazine is commonly used to treat inflammatory bowel disease (Crohn's disease) and rheumatoid arthritis. Sulfasalazine appears to be therapeutic in Crohn's disease because it inhibits the inflammatory response that results from the local cellular destruction in the bowel that is mediated by autoreactive T cells. In rheumatoid arthritis elevated levels of thioredoxin have been found in the synovial fluid of patients suggesting a connection to regulation of cysteine availability in this disease.

A possible mechanism of sulfasalazine is that sulfasalazine and its metabolites (e.g., 5-aminosalicylate, sulfapyridine), and related compounds block cysteine pumps while stimulating glutathione and thioredoxin efflux. This could alter the extracellular thiol balance in the cell.

In various embodiments, the amount of agent that inhibits the x_{c}⁻ transporter is administered at a dose of from about 200 mg to 3 grams/day. In various embodiments, the amount of agent that inhibits the x_{c}⁻ transporter is administered at a dose of from about 10 to about 100 mg/kg/day or from about 200 mg to 3 grams/day. In various embodiments, the amount of agent that inhibits the xc− transporter is administered at a dose of from about 10 to 1000 mg/kg/day, from about 10 to 500 mg/kg/day, from about 500 to 2500 mg/kg/day, or from about 1000 to 3000 mg/kg/day.

Administration of diffusible small thiol compounds in the regimens and doses described above is also contemplated. Administration of diffusible small thiol compounds may also be carried out according to protocols currently in use by physicians in other indications for which small thiol compounds may be used.

Administration may continue for at least 3 months, 6 months, 9 months, 1 year, 2 years, or more.

### Combination Therapy

Therapeutic compositions described herein can also be administered in combination with adjunct therapy used in treatment of excitotoxicity and neurodegenerative diseases, such as antipsychotics, antidepressants, vesicular monoamine transporter (VMAT)-inhibitors such as tetrabenazine, dopamine inhibitors, laquinimod, CNS-immunomodulators, neuroprotective factors, BDNF and agents that upregulate BDNF, ampakines, positive modulators of AMPA-type glutamate receptors, activators of BDNF receptor TrkB and gene therapy.

Antidepressants include: SSRI antidepressants, such as fluoxetine, citalopram and paroxetine, tricyclic antidepressants, such as amitriptyline, other types of antidepressants, including mirtazapine, duloxetine and venlafaxine.

Antipsychotic medication includes risperidone, olanzapine, aripiprazole, tiapride and quetiapine, benzodiazepines, such as clonazepam and diazepam, and mood stabilizers, such as carbamazepine.

In some embodiments, the methods (or uses) described herein further comprise administering a further therapeutic agent selected from the group consisting of tetrabenazine, laquinimod, BDNF, ampakines, fluoxetine, citalopram, paroxetine, amitriptyline, mirtazapine, duloxetine, venlafaxine, risperidone, olanzapine, aripiprazole, tiapride, quetiapine, clonazepam diazepam and carbamazepine.

The cysteamine product and other drugs/therapies can be administered in combination either simultaneously in a single composition or in separate compositions. Alternatively, the administration is sequential. Simultaneous administration is achieved by administering a single composition or pharmacological protein formulation that includes both the cysteamine product and other therapeutic agent(s). Alternatively, the other therapeutic agent(s) are taken separately at about the same time as a pharmacological formulation (e.g., tablet, injection or drink) of the cysteamine product.

In various alternatives, administration of the cysteamine product can precede or follow administration of the other therapeutic agent(s) by intervals ranging from minutes to hours. For example, in various embodiments, it is further contemplated that the agents are administered in a separate formulation and administered concurrently, with concurrently referring to agents given within 30 minutes of each other.

In embodiments where the other therapeutic agent(s) and the cysteamine product are administered separately, one would generally ensure that the cysteamine product and the other therapeutic agent(s) are administered within an appropriate time of one another so that both the cysteamine product and the other therapeutic agent(s) can exert, synergistically or additively, a beneficial effect on the patient. For example, in various embodiments the cysteamine product is administered within about 0.5-6 hours (before or after) of the other therapeutic agent(s). In various embodiments, the cysteamine product is administered within about 1 hour (before or after) of the other therapeutic agent(s).

In another aspect, the agent that inhibits x_{c}⁻ is administered prior to administration of the cysteamine composition. Prior administration refers to administration of the agent that inhibits x_{c}⁻ within the range of one week prior to treatment with cysteamine, up to 30 minutes before administration of cysteamine. It is further contemplated that the agent that inhibits x_{c}⁻ is administered subsequent to administration of the cysteamine composition. Subsequent administration is meant to describe administration from 30 minutes after cysteamine treatment up to one week after cysteamine administration.

In various embodiments, the effects of cysteamine products in combination with an agent that inhibits x_{c}⁻ on the symptoms of the excitotoxicity disease or disorder as described herein are measured as improvements in disease symptoms described above, or are measured as a slowing or decrease in the time of progression of a disease symptom, e.g., a slowed progression of total motor score can be considered an improvement in a disease symptom.

### Kits

The disclosure also provides kits for carrying out the methods of the disclosure. In various embodiments, the kit contains, e.g., bottles, vials, ampoules, tubes, cartridges and/or syringes that comprise a liquid (e.g., sterile injectable) formulation or a solid (e.g., lyophilized) formulation. The kits can also contain pharmaceutically acceptable vehicles or carriers (e.g., solvents, solutions and/or buffers) for reconstituting a solid (e.g., lyophilized) formulation into a solution or suspension for administration (e.g., by injection), including without limitation reconstituting a lyophilized formulation in a syringe for injection or for diluting concentrate to a lower concentration. Furthermore, extemporaneous injection solutions and suspensions can be prepared from, e.g., sterile powder, granules, or tablets comprising a cysteamine product-containing composition and/or a composition comprising an inhibitor of x_{c}⁻ transporter. The kits can also include dispensing devices, such as aerosol or injection dispensing devices, pen injectors, autoinjectors, needleless injectors, syringes, and/or needles. In various embodiments, the kit also provides an oral dosage form, e.g., a tablet or capsule or other oral formulation described herein, of the cysteamine product for use in the method. The kit also provides instructions for use.

While the disclosure has been described in conjunction with specific embodiments thereof, the foregoing description as well as the examples which follow are intended to illustrate and not limit the scope of the disclosure. Other aspects, advantages and modifications within the scope of the disclosure will be apparent to those skilled in the art.

### EXAMPLES

### Example 1

### Cysteamine Reduces Gutamate Cytotoxicity in Striatal Neurons

In order to assess the effects of cysteamine on glutamate toxicity, cysteamine was administered to a Huntington's Disease modified cell line.

All cell culture methods were carried out under sterile conditions in a class II laminar flow cabinet. The immortalized cell lines, ST HDH Q111/111 and ST HDH Q7/7 were derived from striatal neurons from HdhQ111/Q111 and HdhQ7/Q7 mice (expressing 111 and 7 glutamine repeats, respectively) and were purchased from Coriell. Cells were cultured in Dulbecco's Modified Eagle Medium (DMEM) supplemented with 10% FBS, 4 mM L-alanyl-L-glutamine (Corning Glutagro), and 400 μg/mL G418. Cells were grown at 33° C. in a 5% CO₂ incubator. All experiments used cells with passages lower than 14.

Cells were plated at a density of 8×10³ or 1.2×10³ cells/well (CellTox or XTT, respectively) in sterile 96 well plates (100 uL). Cells were allowed to adhere overnight at 33° C. in a 5% CO₂ incubator. Test compounds were applied and left to incubate for an additional 24 hours. Membrane integrity was assessed by staining with CellTox Green (Promega) reagent following manufacturer guidelines for the Endpoint (2×) Method. Viability was assessed using an XTT Cell Proliferation Kit (ATCC). Before adding XTT reagent, wells were aspirated and washed with serum free media. 50 uL of XTT reagent and 100 uL serum free media were then added to each well and incubated for 2-4 hours before acquiring data.

Cells were seeded into sterile 12 well plates at an appropriate density (80-120 k cells/well) to reach 50-75% confluence after 24 hours. Cells were allowed to adhere overnight at 33° C. in a 5% CO₂ incubator. Thereafter, wells were treated with test compounds. Following incubation with the compounds for 24 or 48 hours, wells were rinsed with DPBS, and detached using 500 uL Accutase (EMD). Pellets were rinsed and re-suspended in 250 uL DPBS and live cells were counted on a Cellometer Auto 2000.

Test compounds include: Gamma-fluorobenzylproline, GFBP (Sigma), which blocks the Alanine-Serine-Cysteine transporter (ASCT); (S)-4-carboxyphenylglycine, 4CPG (R&D), which blocks the amino acid antiporter, Xc−, which mediates the exchange of extracellular L-cystine and intracellular L-glutamate; 2-aminobicyclo-(2,2,1)-heptane-2-carboxylic acid, BCH (Sigma), a blocker of System L inhibitor, which transports neutral amino acids and mixed disulfides; Sulfasalazine (Sigma), which blocks the amino acid antiporter, Xc−; N-Methyl-D-aspartic acid, NMDA (Sigma), which activates the NMDA Receptor; L-Buthionine-sulfoximine, BSO (Sigma), which reduces levels of glutathione by inhibiting gamma-glutamylcysteine synthetase, the enzyme required in the first step of glutathione synthesis; L-Glutamic acid, G (Sigma) which is an excitotoxicity neurotransmitter; and 2-aminoethanethiol or cysteamine, CSH (Pierce).

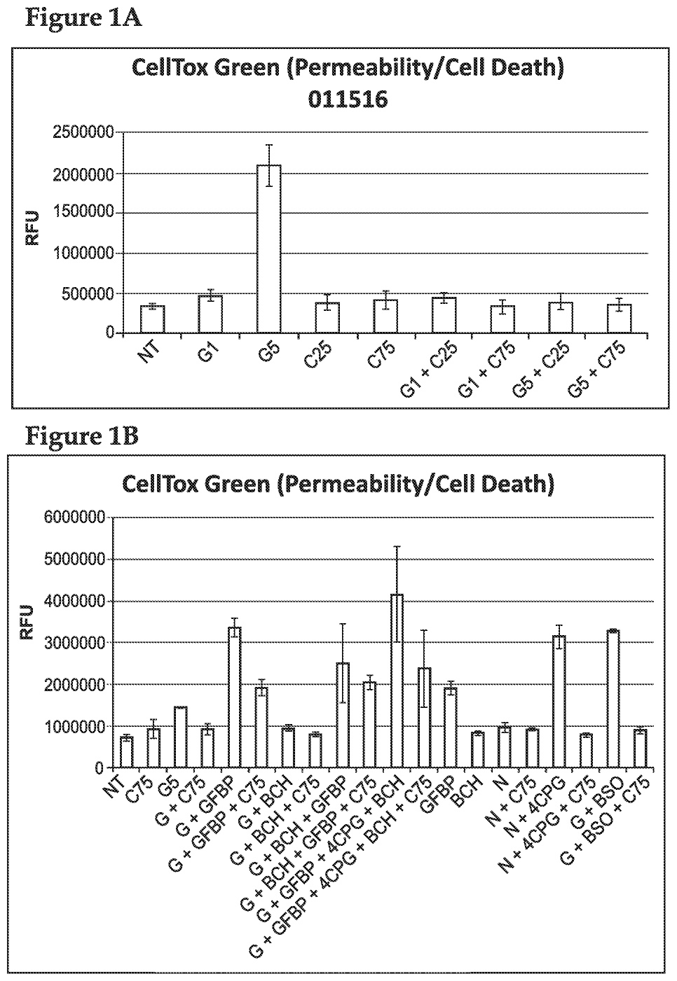

In a Permeability/Cell Death assay based on Cell Tox Green, 5 mM glutamate was toxic to cells when given alone, but cytotoxicity was reduced to control levels when given in combination with cysteamine at either 25 μM or 75 μM (FIG. 1A). Glutamate toxicity was also induced and cells cultured with the test agents above to determine if other agents have an effect of reducing glutamate toxicity. Use of cysteamine in combination with other test agents such as GFPB (5 mM), and NMDA (500 uM)+4CPG (250 uM) or BSO (250 uM) was also able to reduce sensitivity of cells to toxicity induced by glutamate and/or the test agents. (FIG. 1B).

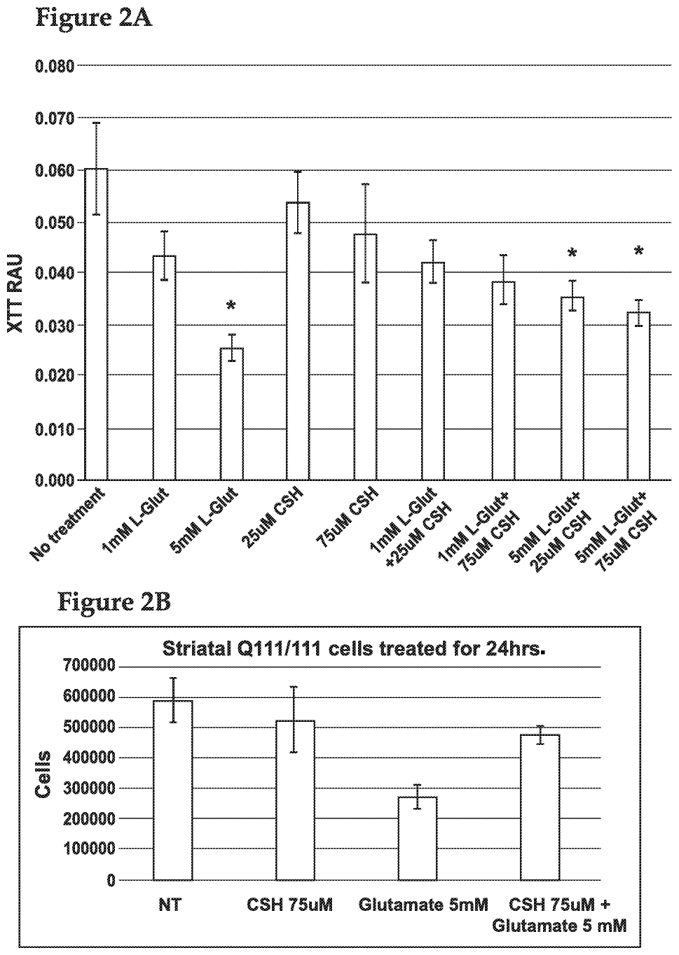

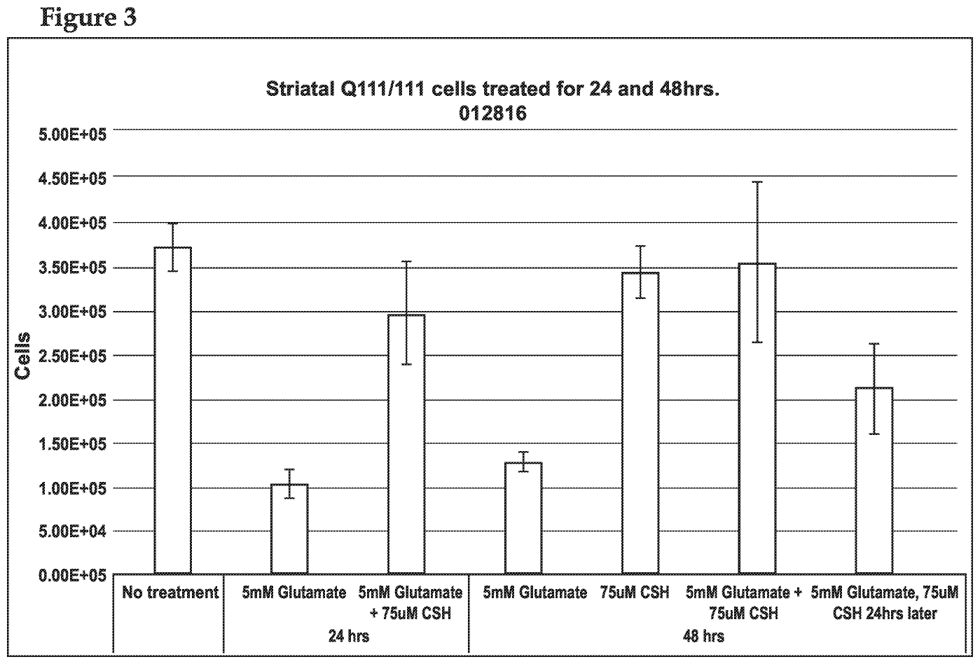

In an XTT Cell Proliferation assay, HDH Q111/111 cells experience glutamate excitotoxicity, however cysteamine in culture at 25 and 75 μM is able to rescue glutamate-induced excitotoxicity (FIGS. 2A and 2B). When striatal cells were cultured with glutamate and cysteamine was administered 24 hours later, the positive effects of cysteamine on the cell viability was still observed (FIG. 3).

In order to determine the effects of glutathione on cell rescue, HDH cells were incubated in in the presence of cysteamine and/or buthionine sulfoxime (BSO), which reduces levels of glutathione by inhibiting glutathione synthesis, and cell viability was determined by CellTox Green. Culture of cells in the presence of cysteamine and BSO reduced the number of dead cells compared to culture with BSO alone, indicating that cysteamine can rescue cells from cytotoxicity in the absence of glutathione.

### Example 2

### Assessment of Cysteamine on Gutamate Cytotoxicity In Vivo

In order to test the effects of cystemaine in combination with an inhibitor of the x_{c}⁻ antiporter, animal models of neurodegenerative diseases are employed.

For example, R62 Huntington's Disease mice are administered cysteamine at 225 mg/kg, 100 mg/kg, or 50 mg/kg daily for 7 days, alternatively in combination with sulfasalazine or another xc− inhibitor, at 50 mg/kg, 100 mg/kg, 150 mg/kg or 250 mg/kg daily or as determined to be effective, and measurement of brain activity and other readouts of Huntington's Disease are determined. Mice are also treated for 8 weeks with cysteamine plus x_{c}⁻ inhibitor and various neurological tests performed during the treatment period, including, rotarod test (4, 6, 8, 10 weeks), neurological index (11 wks), open field test (4, 6, 8, 10, 12 wks), 2 choice swim test (9 wks), gait analysis (11 wks) and MRI (12 wks). Biomarkers such as BDNF levels, and neuronal or glial cell markers are also assessed to determine the effects of treatment on cell morphology and composition.

The Q175 model for Huntington's Disease is also contemplated for use to measure efficacy of the treatment. Animal models for stroke, ischemia, Parkinson's Disease, Alzheimer's Disease, ALS, Multiple Sclerosis and other neurodegenerative diseases are also known in the art and can be used to assess the therapeutic efficacy of cysteamine and xc− inhibitor in these diseases.

Numerous modifications and variations in the invention as set forth in the above illustrative examples are expected to occur to those skilled in the art. Consequently only such limitations as appear in the appended claims should be placed on the invention.

1. A method for treating a subject having an excitotoxicity disorder comprising administering an effective amount of a cysteamine composition in combination with an agent that blocks activity of glutamate/cystine antiporter x_{c}⁻.

2. A method for slowing the degeneration of neurons in a subject comprising administering an effective amount of a cysteamine composition in combination with an agent that blocks glutamate/cystine antiporter x_{c}⁻.

3. A method for treating or ameliorating glutamate toxicity in a subject comprising administering an effective amount of a cysteamine composition in combination with an agent that blocks glutamate/cystine antiporter x_{c}⁻.

4. The method of claim 1 wherein the administering reduces neuronal glutamate toxicity.

5. The method of claim 1 wherein the agent that blocks x_{c}⁻ is selected from the group consisting of sulfasalazine, 4-s-carboxyphenylglycine, 4-s-sulfonylphenylglycine, sorafenib, erastin, and [(R,S)-4-[4′-carboxyphenyl]-phenylglycine.

6. The method of claim 1 wherein the excitotoxicity disorder is selected from the group consisting of spinal cord injury, stroke, traumatic brain injury, chronic traumatic encephalopathy (CTE), hearing loss, neurodegenerative diseases, multiple sclerosis, Alzheimer's disease, amyotrophic lateral sclerosis (ALS), Parkinson's disease, Huntington's disease, concussion, and CNS depressant-withdrawal syndrome.

7. The method of claim 1 wherein the amount of cysteamine composition is from about 1 to about 50 mg/kg/day.

8. The method of claim 1 wherein the agent is sulfasalazine.

9. The method of claim 8 wherein the amount of sulfasalazine is from about 10 to about 100 mg/kg/day.

10. The method of claim 1 wherein striatal neuron damage is reduced in the subject compared to subjects not receiving the cysteamine composition and agent that blocks glutamate/cystine antiporter x_{c}⁻.

11. The method of claim 1 wherein the cysteamine composition is given prior to the agent that blocks x_{c}⁻, concurrently with the agent that blocks x_{c}⁻ or after the agent that blocks x_{c}⁻.

12. The method of claim 1 wherein the administering improves one or more symptoms total motor score, mobility, cognitive ability, or other symptom of an excitotoxicity disorder.

13. The method of claim 1 wherein one or more symptom includes total motor score, mobility, cognitive ability, or other symptom of an excitotoxicity disorder.

14. The method of claim 1 wherein the excitotoxicity disorder is Huntington's Disease.

15. The method of claim 1 wherein the excitotoxicity disorder is Alzheimer's Disease.

16. The method of claim 1, wherein the cysteamine composition and/or agent that blocks x_{c}⁻ further comprises a pharmaceutically acceptable carrier.

17. The method of claim 1, wherein the cysteamine composition and/or agent that blocks x_{c}⁻ is formulated as a sterile pharmaceutical composition.

18. The method of claim 1, wherein the method comprises administering cysteamine or a pharmaceutically acceptable salt thereof.

19. The method of claim 18, wherein the salt is cysteamine bitartrate.
